(19) United States
(12) Patent Application Publication
(10) Pub. No.: US 2012/0041770 A1
(43) Pub. Date: Feb. 16, 2012

(54) SYSTEMS, METHODS, AND APPARATUS FOR PROVISIONING AND TRACKING MEDICAL SUPPLIES FOR MEDICAL PROCEDURES

(76) Inventor: Richard Philippe, Laval (CA)

(21) Appl. No.: 12/853,709

(22) Filed: Aug. 10, 2010

(51) Int. Cl. G06Q 50/00; G06Q 10/00; G06Q 30/00

(52) U.S. Cl. 705/2

(57) ABSTRACT

According to one aspect, a system for provisioning medical supplies for medical procedures. The system includes at least one data storage device for storing data related to the provisioning of medical supplies, and at least one processor operably connected to the at least one data storage device. The at least one processor is configured to, for at least one medical procedure, generate a pick list including predictable medical supplies for that procedure based on a template for that procedure, receive data about predictable medical supplies picked at one or more storage locations for that procedure based on the pick list and store that data in the at least one data storage device, and receive data about unpredictable medical supplies picked at the one or more storage locations and store that data in the at least one data storage device.

### FIELD

Embodiments described herein are directed to the provisioning of medical supplies and in particular to systems, methods and apparatus for provisioning medical supplies for specific procedures and tracking medical supplies associated with those medical procedures.

### SUMMARY

According to one aspect there is provided a system for provisioning medical supplies for medical procedures. The system comprises at least one data storage device for storing data related to the provisioning of medical supplies, and at least one processor operably connected to the at least one data storage device. The at least one processor is configured to, for at least one medical procedure, generate a pick list including predictable medical supplies for that procedure based on a template for that procedure, receive data about predictable medical supplies picked at one or more storage locations for that procedure based on the pick list and store that data in the at least one data storage device, and receive data about unpredictable medical supplies picked at the one or more storage locations and store that data in the at least one data storage device.

In some embodiments, the at least one processor is further configured to update the template for that procedure based on the received data about unpredictable supplies.

In some embodiments, the at least one processor is further configured to receive data about unused medical supplies after the procedure is complete, store that data in the at least one data storage device, and update the template based on the received data about the unused medical supplies.

In some embodiments, the at least one processor is configured to customize the pick list for each procedure based on preference data for that procedure stored in the at least one data storage device.

The at least one location may include a first location where at least one of the predictable medical supplies is picked based on the pick list. The at least one location may include a second location where at least one of the unpredictable medical supplies is picked before the procedure. The at least one location may include a third location where at least one of the unpredictable medical supplies is picked during the procedure.

The system may further comprise at least one electronic data collection device for collecting data about at least one of the unpredictable medical supplies being picked and the unused medical supplies being returned, the electronic data collection device operable to transmit the collected data to the at least one processor for storing in the at least one data storage device.

The at least one processor may be operable to update patient information based on at least one of the pick list and the unused medical supplies after the procedure is complete. The patient information may include financial information related to supplies consumed during the procedure. The financial information may include at least one of billing information for charge capture and case costing information. The patient information may include medical information about at least one uniquely identifiable medical supply consumed during the procedure.

According to another aspect, there is provided a method of provisioning medical supplies for medical procedures. The method includes generating a pick list using at least one processor, the pick list including predictable medical supplies for a particular procedure based on a template for that procedure, receiving data about predictable medical supplies picked at one or more storage locations for that particular procedure based on the pick list and storing that data in at least one data storage device, and receiving data about unpredictable medical supplies picked at one or more storage locations for that predictable procedure and storing that data in at least one data storage device.

The method may include updating the template for that procedure based on the received data about unpredictable supplies.

The method may further comprise receiving data about unused medical supplies after the procedure is complete, storing that data in the at least one data storage device, and updating the template based on the received data about the unused medical supplies.

The method may further comprise customizing the pick list for each procedure based on preference data for that procedure stored in the at least one data storage device.

The method may further comprise updating patient information based on at least one of the pick list and the unused medical supplies after the procedure is complete.

### DRAWINGS

The embodiments herein will now be described, by way of example only, with reference to the following drawings, in which:

FIG. 1 is a block diagram illustrating a system for tracking medical supplies according to one embodiment;

FIG. 2 is a block diagram illustrating a method for tracking medical supplies according to another embodiment; and

FIG. 3 is a schematic diagram of a pick list for use with the system of FIG. 1 and method of FIG. 2 according to another embodiment.

### DESCRIPTION OF VARIOUS EMBODIMENTS

Medical facilities such as hospitals are often divided into multiple departments, each of which may relate to a particular specialization. Each department may use general and specific supplies when performing various different procedures (e.g. surgical procedures). These supplies are often stored in different storerooms within the various departments, in centralized warehouses that may be located within or adjacent the medical facility, and/or at different physical locations.

General supplies could include common articles such as bandages or cleaning rags that are commonly used in many different types of procedures, while specific supplies could include specialized equipment (e.g. scalpels) or pharmaceutical drugs (e.g. a particular anesthetic) that are more specific to the type of procedure being performed. Some supplies may be expensive “high value” items, such as medical implants, pacemakers, and so on. Other supplies may be relatively inexpensive, such as gloves or bandages.

When preparing for a particular procedure, it is generally necessary to collect supplies from the storerooms and warehouses and provide the supplies to a location where the procedure will be carried out (e.g. an operating room). Generally, this may be done by collecting supplies and placing them onto a “case cart”, which may then be placed in the operating room for use during the procedure.

In some instances, the types and quantities of supplies needed for a particular procedure may vary based on a number of different factors. For example, some supplies may be “predictable” supplies that can be determined well in advance of a specific procedure. Other supplies may be unpredictable “pre-case” or “pre-procedure” supplies that may be unknown until shortly before the procedure occurs (e.g. the specific sizes of gloves for nurses that will be in the operating room during the procedure).

Other supplies may be unpredictable “in-case” or “in-procedure” supplies that are only identified as the procedure unfolds. For example, in some cases, in-procedure supplies could include unique high value items such as pacemakers or medical implants. In other instances, the need for in-procedure supplies may arise due to unforeseen events, such as unexpected complications during surgery.

Predictable supplies are normally based on requirements that are common for all or a majority of instances of a particular event or type of procedure. For example, when performing any type of surgery, it may be predicted that certain supplies will always be required (e.g. a sheet for covering the operating table, bandages, gauze, etc.). Furthermore, when performing a specific type of surgery (e.g. an appendectomy), it may be the case that a specific type of equipment will always be used (e.g. a certain clamp, a rib-spreader, etc.).

These predictable supplies can normally be picked in advance of the particular procedure depending on the operating schedule. For example, in some cases predictable supplies may be picked the day before the particular procedure or a few hours before the procedure is scheduled to occur. As such, case carts with these supplies can be prepared when nurses or other highly-skilled personnel have down time, or be prepared by lesser-skilled personnel based on a list of the predictable supplies.

However, unpredictable supplies normally cannot be picked until shortly before the procedure (e.g. within a few hours or few minutes for pre-procedure supplies) or even during the procedure itself (e.g. for in-procedure supplies). In particular, it is often the case that pre-procedure supplies can be determined only once certain details about the specific doctor, nursing staff, or patient are known. For example, the patient may have an allergy to certain anesthetics, the nursing staff may need gloves of specific sizes, and so on.

Unpredictable in-procedure supplies, on the on the other hand, can normally only be picked during the procedure. For example, the need for in-procedure supplies may arise due to unforeseen compilations that arise during the procedure. In other instances, the operation must be underway before the surgeon can determine which particular in-procedure supply is needed (e.g. what model or size of a particular medical implant, such as a hip replacement joint, is suitable for use with a particular patient).

Because many supplies are unpredictable, generally it is difficult to select more than 80% of the supplies required for a procedure well in advance of that procedure. That is, generally, about 80% of the necessary supplies for a particular procedure may be predictable supplies that can be selected well in advance of that procedure, about 15% of the necessary supplies are pre-procedure supplies that can only be predicted and picked shortly before the procedure begins, and the rest of the necessary supplies (e.g. about 5%) are in-procedure supplies that can only be identified during the procedure (and cannot be selected in advance of the procedure).

In some cases, this percentage breakdown may vary depending on various factors such as the type of procedure, the specialty of the medical professionals performing the procedure, and the accuracy of other information available (such as patient information).

Generally, it is preferable that a higher percentage of the supplies necessary for a particular procedure are picked by lesser-skilled personnel to optimize allocation of the time of the highly-skilled personnel. Documenting information about features such as the product code, description, exact location and quantity necessary for a procedure may facilitate efficient delegation of picking predictable items to lesser-skilled personnel. However, accurate information about what predictable supplies are necessary may not be available or may not be up-to-date, which often means that highly-skilled personnel perform these picking tasks. This is undesirable, as it is more efficient for such tasks to be delegated to lesser-skilled individuals while highly-skilled medical personnel can focus on medical tasks.

Accordingly, one of the challenges in the medical supplies industry is the provisioning of medical supplies for specific procedures in an efficient manner.

A further challenge is presented in tracking unique items, typically high value or expensive supplies, such as medical implants, pacemakers, etc. that are often only uniquely identified during the procedure. During a procedure, particularly when highly-skilled personnel are busy performing the procedure, finding time to adequately document which particular high value item has been selected (e.g. the serial number of the pacemaker or implant that has been inserted into a patient) can be difficult, and errors in data entry tend to be common. For example, an operation may be underway on a particular patient before the appropriate size of hip-joint implant can be identified and inserted into a patient.

Tracking such unique items may be important for various reasons. For example, tracking can be important in ensuring that proper items are billed to a particular patient. Tracking may also be used to perform various financial analyses, such as case costing. Furthermore, tracking unique items may be important to ensuring that the medical records of the patient are properly updated.

After a particular procedure has been completed, unused supplies should be returned to inventory, ensuring that the patient is not charged for items that were not actually used during the procedure to ensure an accurate case cost and avoid over-billing the client. This can be a time-consuming process, and accurately tracking returned inventory can be cumbersome and unwieldy.

Also, it may be desirable to update the information about the supplies used for a procedure to improve the accuracy of available information for subsequent cases.

It will be appreciated that numerous specific details are set forth herein in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of various embodiments as described herein.

In some cases, the embodiments of the systems and methods described herein may be implemented in hardware or software, or combinations of both. Some embodiments may be implemented in computer programs executing on programmable computers each comprising at least one processor, a data storage device (which may include volatile and non-volatile memory and/or other storage elements), at least one input device, and at least one output device. For example and without limitation, the programmable computers may be a mainframe computer, server, personal computer, laptop, personal data assistant, tablet computer or cellular telephone, including one or more processors. Program code may be applied to input data to perform the functions described herein and generate output information. The output information may be applied to one or more output devices in known fashions.

Each program may be implemented in a high level procedural or object oriented programming and/or scripting language to communicate with a computer hardware system. However, the programs can be implemented in assembly or machine language, if desired. In any case, the language may be a compiled or interpreted language. Each such computer program may be stored on a storage media or a device (e.g. read only memory (ROM) or magnetic diskette) readable by a general or special purpose programmable computer, for configuring and operating the computer when the storage media or device is read by the computer to perform the procedures described herein. The systems and methods may also be considered to be implemented as a non-transitory computer-readable storage medium, configured with a computer program, wherein the storage medium so configured causes a computer to operate in a specific and defined manner to perform at least some of the functions described herein.

According to some embodiments, the systems and methods as described herein may provide at least some of the following functions.

The first function generally relates to data collection. During a data collection function, a library or record of information can be generated and stored in a supply management module. This information can include details about what supplies are used for various medical procedures that are performed at a medical facility.

This data collection may be done by observing and recording actual provisioning of supplies for actual procedures. For example, when nurses and other skilled-staff are selecting unpredictable pre-procedure supplies, they may be monitored or observed and the data indicative of the actions recorded (in some cases manually, in other cases using electronic aids such as bar code readers or RFID tags and readers, or other electronic handheld communication devices).

Furthermore, the data collection may track not only the types of procedure being performed, but also other information about each procedure, such as what surgeon performed the operation, what nursing staff assisted, and so on.

The second function generally relates to generating a preference card or template based on the collected data. For various combinations and sub-combinations of procedures, surgeons, and other details, preference data or “preference cards” may be generated. Each preference card can include a prediction of the supplies that will be required for a particular procedure based on the data collected above and other criteria that may be relevant to the operation (e.g. which doctor will be performing the procedure). In particular, by collecting information about actual procedures, more accurate predictions can hopefully be made about the supplies needed for subsequent procedures based on the preferences of the various surgeons, nursing teams and so on.

In effect, the preference cards or templates represent improved (and in some cases continuously updated) predictions that are used in an attempt to increase the percentage of supplies that can be treated as “predictable” supplies, and thus be picked well in advance of the actual procedure. These preference cards or preference data can be stored in the supply management module (e.g. in one or more data storage devices) and accessed as desired.

Third, when a specific procedure is actually scheduled, a specific pick list for that procedure can be generated based on the preference card or template for that specific procedure, and which can be based on details such as the surgeon, the nursing team, patient details, and so on.

This pick list can then be used to pick the predictable supplies and place them in a case cart (with the pick list being updated as the picking progresses). In particular, by providing for a pick list with better predictions about supplies, more of the picking may be done by lesser-skilled personnel in advance of the particular procedure being performed.

The pick list may also be used to track picking the unpredictable pre-procedure supplies, as well as in-procedure supplies consumed as the procedure progresses. In some embodiments, the pick list may be provided with a unique identifier (which may be represented as a bar code or a RFID tag) that allows information on the pick list to be communicated to the supply management module and stored in one or more data storage devices as picking proceeds. In some embodiments, the pick list may be dynamic in that it can be updated “on-the-fly” as more accurate predictions are made for a particular procedure (e.g. as further details are learned about the patient, about the personnel assisting with the procedure, and so on).

The fourth function relates to returning unused supplies to inventory. Once a particular procedure is complete, any supplies that have not been consumed should be returned to inventory. In some embodiments, this can be done by using the pick list to record items as they are returned.

Once returns are completed, the pick list can be used to generate patient information related to that particular procedure, such as billing information or medical information about what supplies were used, what unique items were inserted into the patient (e.g. implant serial numbers), and so on.

The fifth function relates to data refinement. After each procedure is complete, data about the actual supplies consumed can be compared to the preference card or template used to generate the pick list, and any differences can be used to modify the template. Over time, the preference data or templates for specific procedural events can be updated based on the actual supplies that are consumed, and this feedback can hopefully be used to generate better initial predictions about the supplies that will be required for subsequent procedures. In this manner, the supply management module can “learn” to provide improved predictions as more and more data is collected.

Accordingly, using systems and methods as described herein it may be possible to perform at least some of improving the data capture related to medical supplies, building a library or database with data records of preference cards, tracking the picking of supplies at various stages (including before, during and after a procedure), and continually updating preference card or template information after procedures are completed.

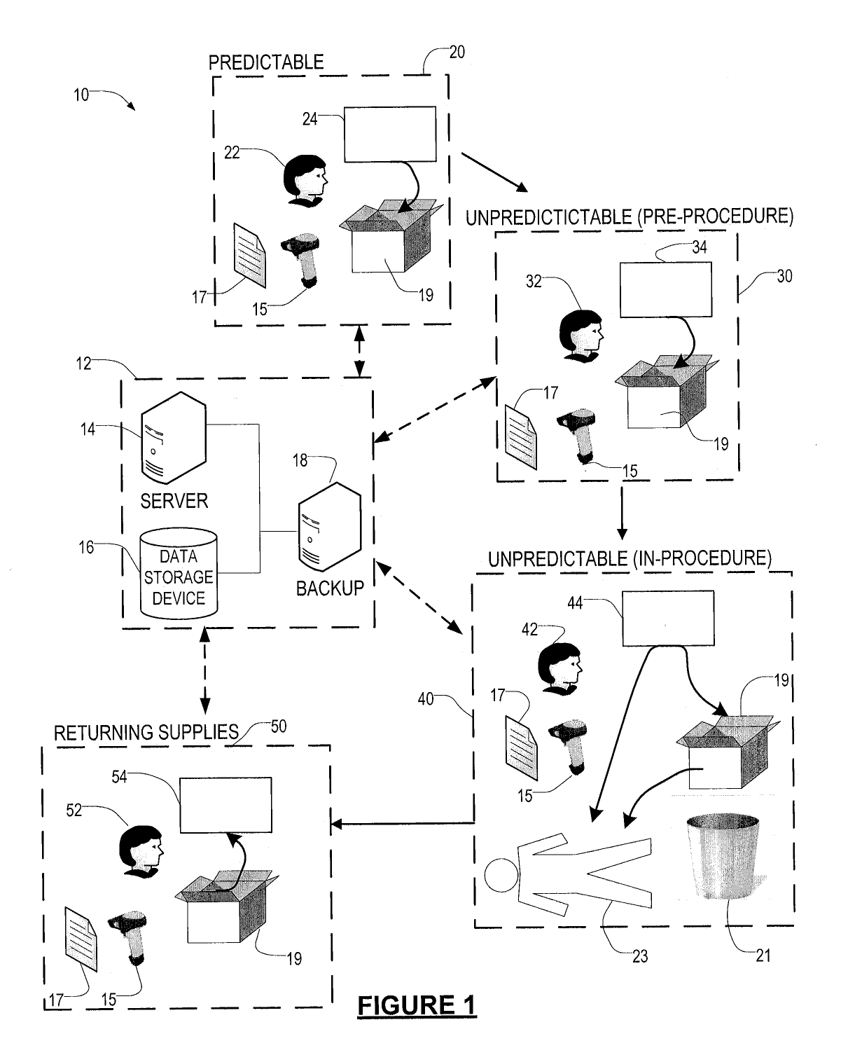

Referring now to FIG. 1, illustrated therein is a system 10 for provisioning and tracking medical supplies for medical procedures according to one embodiment. The system 10 generally includes a supply management module 12, which includes at least one server 14 and at least one data storage device 16 operatively coupled to the at least one server 14.

The at least one data storage device 16 could be one or more databases or other memory modules for storing data related to the provisioning of medical supplies, which can include preference card or template data but may also include other information such as patient information, inventory information about quantities of medical supplies, location information about where specific medical supplies are located at the medical facility, billing information, and so on.

In some embodiments, the data storage device 16 may include a spreadsheet, a relational database, or any other type of information storage for storing information assisting in the provisioning of supplies. In some cases, the at least one data storage device 16 may include one or more of the following information: the Common User Product (CUP) number, the Device Identification Number (DIN) of the supplies, descriptions of the supplies, data on the corresponding RFID tags or bar codes associated with the supplies, an electronic signal corresponding to voice input of the CUP or DIN, location information (department, room number, rack identification, row number, etc.) and so on.

The at least one server 14 generally includes at least one processor. The processor is operable for sending instructions to, and receiving data from, the at least one data storage device 16, as well as for communicating with other modules within the medical facility, receiving information from the various pick lists, electronic data collection devices (e.g. bar code readers, RFID readers, and so on).

The supply management module 12 may also include other components, such as a backup module 18 which may duplicate at least some of the data in the data storage device 16 (e.g. for disaster recovery purposes). The backup module 18 could be located at the same physical location, or at a different physical location as the server 14 and data storage device 16.

Generally, supply management module 12 is operable to receive a notification that a particular procedure will be performed. The supply management module 12 then generates a pick list 17 for that procedure based on data such as the preference card or template data, patient information, and other details. The pick list 17 may then be sent to one or more locations so that various supplies can be prepared in advance of the procedure.

For example, as shown in FIG. 1 the pick list 17 may be provided to a first location 20 for picking predictable supplies. At this location, a first user 22 selects medical supplies from a supply depot 24 (e.g. a shelf, cabinet, etc.) and then adds those supplies to a case cart 19.

In some embodiments, the first user 22 need not be a nurse or other highly-skilled medical personnel, but could be a lesser-skilled worker who follows instructions on the pick list 17 to select the appropriate supplies and add them to the case cart 19.

As supplies are added to the case cart 19, the first user 22 may use an electronic data collection device 15 (e.g. a barcode or RFID reader) to scan the items and update the pick list 17. In some embodiments, this may be done by scanning a barcode or RFID tag on the supply being added, then scanning the pick list 17 and/or the case cart 19 (or vice versa).

Generally, the electronic data collection device 15 may be a wired or a wireless reader. The electronic data collection device 15 receives an input corresponding to a particular supply to be provisioned. In some embodiments, the input may include any one or more of the following: reading a bar code, reading a magnetic card, a keyboard entry, a voice command, a RFID reading, or any other type of electronic input that represents the supply to be provisioned.

Hence, the electronic data collection device 15 may include one or several of the following input modules depending on the type of input used: a bar code reader, a magnetic card reader, a keyboard, a voice command module, an RFID reader, and so on.

The first user 22 uses the reader electronic data collection device 15, which is in some cases a small unit, in the shape of a handheld unit, or having a portion thereof that can be carried by or attached to the user for convenience purposes. Depending on the type of input available, the user 22 can either read the CUP or DIN number on the product/supply or packaging thereof, and place that product in the case cart 19. In some embodiments, the user 22 may also identify the case cart 19 by wirelessly reading an electronically recognizable identification tag (e.g. an RFID tag) on the case cart 19.

Furthermore, in some embodiments, each pick list 17 may be provided with a unique identifier (which could be a serial number) and which could be an electronically recognizable identification tag. For example, the unique identifier could be a bar code that can be read by an optical scanner, or an RFID tag.

In some embodiments, the unique identifier can be associated with a particular patient. Thus, as various supplies are added to a specific case cart 19 for that particular procedure, the unique identifier can be used to track which supplies have been picked (and in some cases this information can be communicated back the supply management module 12, optionally in real time or substantially real-time as the pick list 17 is updated).

In some embodiments, the first location 20 may be at or near the medical facility where the procedure will take place. In other embodiments, the first location 20 may be at a different physical location (e.g. the first location 20 could be a warehouse at a medical supply company offsite).

In some embodiments, the picking at the first location 20 can be done hours or days in advance of the date the particular procedure will be performed.

When the first user 22 has completed adding the predicable supplies to the case cart 19 at the first location 20, the case cart 19 can then be sent a second location 30.

Generally, the case cart 19 and pick list 17 will tend to stay together as the case cart 19 is stocked and then provided to other picking locations and then eventually the operating room. However, in some embodiments the pick list 17 may be sent electronically from location to location while the case cart 19 physically travels between locations.

In some embodiments, the second location 30 may generally be used for picking unpredictable supplies (specifically pre-procedure supplies). For example, the second location 30 could be a “clean room” supply area provided in or adjacent an operating room where the procedure will be taking place. In some instances, the picking in the second location 30 may be performed shortly before the procedure is to take place.

Similar to as described above, a second user 32 selects medical supplies from a supply depot 34 (e.g. a shelf, cabinet, etc.) and then adds those supplies to the case cart 19. In some instances, the second user 32 may select medical supplies based on updates that may have been made to the pick 17 since the case cart 19 left the first location 20. For example, as additional details become known just before the procedure, the pick list 17 can be updated with more supplies to be picked.

In other embodiments, the second user 32 may select supplies at the second location 30 based on other factors (e.g. such as last minute instructions from the doctor, their own personal knowledge, and so on).

In some embodiments, the second user 32 can update the pick list 17 using an electronic data collection device 15 (e.g. a barcode or RFID reader) to scan the items and update the pick list 17 similar to as described above.

In some embodiments, the second user 32 could be nurse or other highly skilled medical personnel. However, in other embodiments the second user 32 may be a lesser-skilled person who may be assigned the role of assisting with supply picking.

As shown, in some embodiments as supplies are picked at the second location 30, information about the supplies being picked may be sent to the supply management module 12 (in some cases in real-time or substantially real-time).

When the second user 32 has completed adding supplies to the case cart 19 at the second location 30, the case cart 19 can then be sent a third location 40. In some embodiments, the third location 40 may be the location where the procedure is to take place, for example an operating room where surgery on a patient 23 will occur.

During the procedure, supplies can be removed from the case cart 19 as needed to prepare for and perform the procedure. For example, some supplies can be used to prepare the third location 40 for the operation (e.g. sheets can be used to cover the operating table, cleaners can be used to clean equipment, certain items may be unpacked so as to be ready for use, and so on). Other supplies will be used as the procedure progresses, such as bandages to pack wounds, and so on.

In some instances, certain unpredictable in-procedure supplies will also be required during the procedure. These could be completely unpredictable (for example, extra supplies required due to unforeseen complications that arise during the procedure) or may be unique items (e.g. implants) where the specific item to be consumed may not be known until the procedure is underway.

Generally, such unpredictable in-procedure supplies may be selected from a supply depot 44 (e.g. a shelf, cabinet, etc.), which could be in the fourth location or which could be located at another location (e.g. in an adjacent “clean room”). Such supplies can then be used directly to treat the patient 23, or may be placed into the case cart 19 before being provided to the patient 23.

In some cases, for such supplies, a third user 42 (which could be a nurse or other personnel in the third location 40) can use an electronic data collection device 15 to update the pick list 17, generally similarly to as described above.

In some embodiments, as supplies are consumed at the fourth location 40 (particularly high-value supplies that are uniquely selected during the procedure e.g. a specific pacemaker or implant), it may be desirable that the supplies are accurately tracked without using the electronic data collection device 15. In such embodiments, this may be done by using a “smart” or “intelligent” receptacle 21 (e.g. a “smart” garbage can). As high-value items are removed from their packaging and consumed, the packaging can be disposed of in the “smart” receptacle 21, which can automatically track electronically recognizable identification tags on the packaging (e.g. bar codes, RFID tags, etc.) and communicate such information to the supply management module 12 (which may be used for tracking, billing purposes, for updating the patient's medical records, and so on).

Once the procedure is complete, the case cart 19 with any remaining supplies can be sent to a fourth location 50 so that the remaining supplies can be returned to inventory. In particular, the patient should not be charged for items that were not consumed during the procedure.

At the fourth location, a fourth user 52 can remove unused supplies from the case cart 19 and return these supplies to a supply depot 54 (e.g. a shelf, cabinet, etc.). As this is done, the pick list 17 can be updated using the electronic data collection device 15 to remove unused items from the list. In some embodiments, the user 52 may be guided by the electronic data collection device 15. For example, the user 52 may use voice commands to find the location for returning the supply items to the depot 54. That is, the user 52 may read the manufacturer product number (partially or completely) to the electronic data collection device 15 which may then direct the user to the location where the particular items are to be returned (e.g. a specific bin on a shelf). The user 52 may also communicate the quantity of supplies being returned from the cart 19 using voice commands such that this information is collected and stored. This information can be communicated to the supply management system 12, which can be used for updating patient information, such as billing information (e.g. to generate an itemized list of all the supplies that were consumed during the particular procedure) or medical history information (e.g. details of any high value items such as implants that were inserted into the patient).

In some embodiments, the fourth location 50 could be the same as the first location 20.

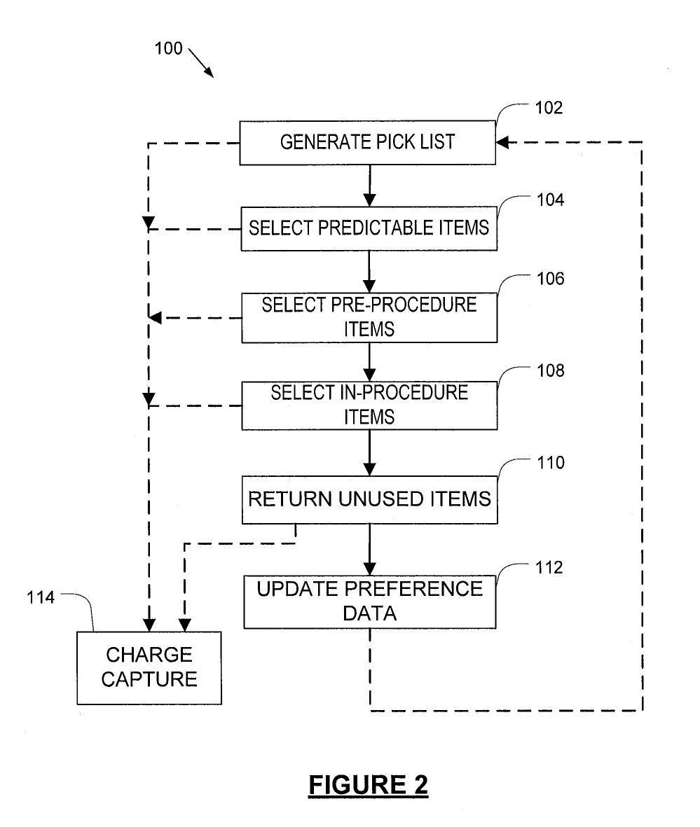

Turning now to FIG. 2, illustrated therein is a method 100 for provisioning medical supplies according to another embodiment.

At step 102, a pick list (e.g. pick list 17) is generated (which could be done by the supply management module 12) for a particular procedure. As discussed above, the pick list may be generated in response to a notification of the procedure to be performed, and may be based on preference data or a template that is known for that particular procedure, as well as other information (such as patient data).

At step 104, predictable medical supply items are selected based on the pick list. In particularly, predictable medical supplies may be picked and placed into a case cart (e.g. case cart 19) based on the pick list 17 (e.g. several hours or days in advance of the procedure).

At step 106, unpredictable pre-procedure items can be selected. For example, shortly before a procedure takes place, other items can be added to the case cart (either based on updates to the pick list or other factors). As this happens, the pick list can be updated.

At step 108, during the procedure, unpredictable in-procedure items can be selected and consumed as needed based on how the procedure progresses. For example, high value items such as implants can be inserted into the patient. As this occurs, information about the high value items may be recorded (and the pick list may be updated accordingly).

At step 110, once the procedure is complete, unused items may be returned to inventory (in some cases with the pick list being updated as this occurs).

At step 112, the actual supplies consumed during the procedure may be sent to a supply management module (e.g. supply management module 12) and used to update preference data or template for that particular procedure. This may allow more accurate estimations to be made of pick lists (e.g. at step 102) so that a larger percentage of items can be picked at step 104 and fewer items must be picked at steps 106 and 108.

At step 114, billing information may be generated based on the various supplies consumed at the various steps in the method 100.

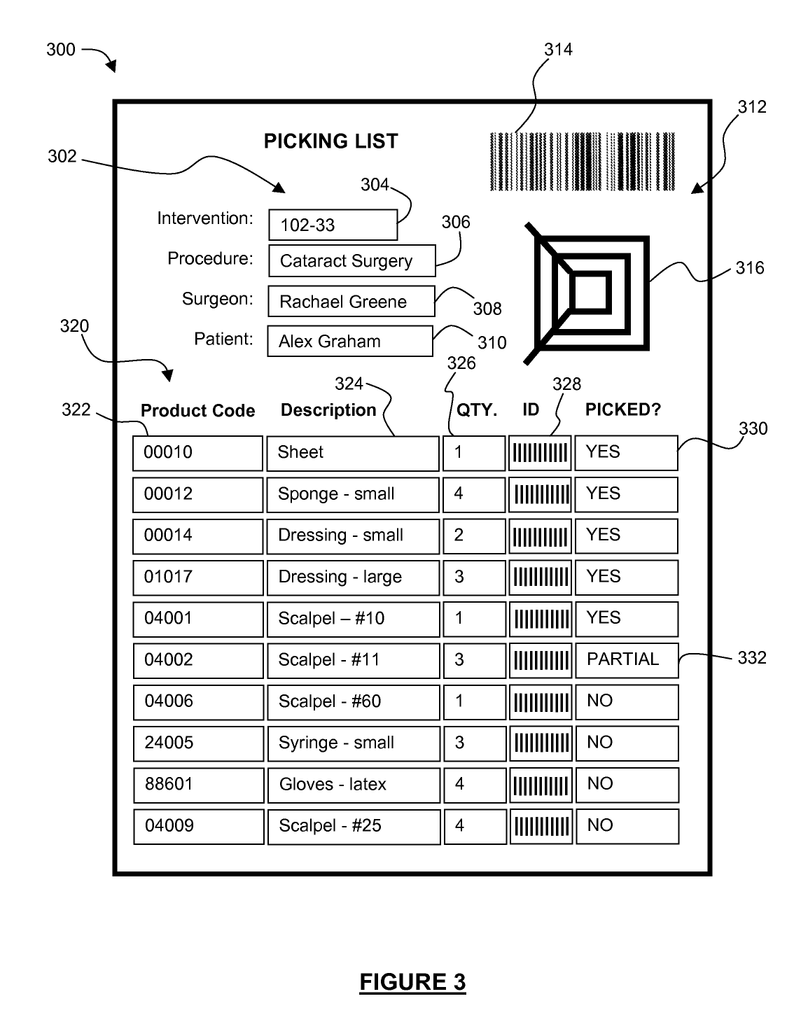

Turning now to FIG. 3, illustrated therein is a pick list 300 according to one embodiment.

As shown, the pick list 300 can include procedure information 302 which could include information specific to that procedure, such as an intervention number 304, a procedure description 306, surgeon information 308 (e.g. the name of the doctor), and patient information 310 (e.g. a patient's name, special notes, etc.).

As shown, the pick list 300 also includes at least one unique identifier 312. In some cases, the unique identifier 312 could include a serial number or other alphanumeric code that may be uniquely associated with a particular pick list 300. In some embodiments, the unique identifier 312 may be represented using at least one of a barcode 314 and an RFID tag 316. The barcode 314 and RFID tag 316 may allow information about each specific pick list 300 to be easily retrieved or updated (e.g. using a handheld barcode reader or an RFID reader).

The pick list 300 also includes supply information indicated generally as 320. The supply information 320 includes information about the specific supplies that should be picked for that specific procedure (as identified by the procedure information 302).

As shown, for each type of item to be picked, the supply information 320 can include information such as a product code 322, a description of the item 324 (e.g. is that item a sponge or a scalpel, size, shape and color information, etc.), a quantity 326 of the item to be picked, a unique identifier 328 (e.g. a barcode) for that particular item, and a status indicator 330 indicating whether that item has been picked. Other supply information 320 could include the location of each particular item, a graphical representation of that item (e.g. a drawing or picture), audio clues that can be triggered to assist the user in locating the item, and so on.

Generally, when a user has the pick list 300, the user can identify and locate one of the items in the supply information 320, physically select that item (e.g. from a shelf or supply cabinet in a storeroom or warehouse) and then place that item into the case cart for that procedure (e.g. case cart 18 shown in FIG. 1). As this occurs, the indicator 330 can be updated to reflect that the particular item has been placed into the case cart.

In some instances, the indicator 330 can be updated by scanning a barcode or RFID tag that is attached to or otherwise associated with the item being picked.

In some instances, the predictable items can be marked automatically such that these items are readily identifiable to the lesser-skilled individual picking them.

In some instances, where multiple quantities of a particular item are required, the indicator 330 may indicate whether the order for that item has been partially filled (as shown in 332).

In some embodiments, the pick list 300 may be presented on a computing device such as a laptop or tablet computer which may be updated dynamically as information about the items picked is updated. In some cases, the computing device may be affixed to the case cart.

In other embodiments, the pick list 300 may be provided as a printed sheet of paper, which may be updated by manually checking the indicator 330.

In some cases, the pick list 300 may be updated as supplies are selected from a warehouse or other storage room at the medical facility.

In some embodiments, a third party medical supplier may provide “pre-assembled” case carts. For example, case carts can be prepared at a different location with at least some supplies being pre-selected, hermetically sealed and shipped to the medical facility for later use.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A system for provisioning medical supplies for medical procedures, comprising:
at least one data storage device for storing data related to the provisioning of medical supplies; and
at least one processor operably connected to the at least one data storage device;
wherein the at least one processor is configured to, for at least one medical procedure:
generate a pick list including predictable medical supplies for that procedure based on a template for that procedure;
receive data about predictable medical supplies picked at one or more storage locations for that procedure based on the pick list and store that data in the at least one data storage device; and
receive data about unpredictable medical supplies picked at the one or more storage locations and store that data in the at least one data storage device.

2. The system of claim 1, wherein the at least one processor is further configured to update the template for that procedure based on the received data about unpredictable supplies.

3. The system of claim 2, wherein the at least one processor is further configured to receive data about unused medical supplies after the procedure is complete, store that data in the at least one data storage device, and update the template based on the received data about the unused medical supplies.

4. The system of claim 1, wherein the at least one processor is configured to customize the pick list for each procedure based on preference data for that procedure stored in the at least one data storage device.

5. The system of claim 1, wherein the at least one location includes a first location where at least one of the predictable medical supplies is picked based on the pick list.

6. The system of claim 5, wherein the at least one location includes a second location where at least one of the unpredictable medical supplies is picked before the procedure.

7. The system of claim 6, wherein the at least one location includes a third location where at least one of the unpredictable medical supplies is picked during the procedure.

8. The system of claim 3 further comprising at least one electronic data collection device for collecting data about at least one of the unpredictable medical supplies being picked and the unused medical supplies being returned, the electronic data collection device operable to transmit the collected data to the at least one processor for storing in the at least one data storage device.

9. The system of claim 3, wherein the at least one processor is operable to update patient information based on at least one of the pick list and the unused medical supplies after the procedure is complete.

10. The system of claim 9, wherein the patient information includes financial information related to supplies consumed during the procedure.

11. The system of claim 10, wherein the financial information includes at least one of billing information for charge capture and case costing information.

12. The system of claim 10, wherein the patient information includes medical information about at least one uniquely identifiable medical supply consumed during the procedure.

13. A method of provisioning medical supplies for medical procedures, comprising:
generating a pick list using at least one processor, the pick list including predictable medical supplies for a particular procedure based on a template for that procedure;
receiving data about predictable medical supplies picked at one or more storage locations for that particular procedure based on the pick list and storing that data in at least one data storage device; and
receiving data about unpredictable medical supplies picked at one or more storage locations for that predictable procedure and storing that data in at least one data storage device.

14. The method of claim 13, further comprising updating the template for that procedure based on the received data about unpredictable supplies.

15. The method of claim 14, further comprising:
receiving data about unused medical supplies after the procedure is complete;
storing that data in the at least one data storage device; and
updating the template based on the received data about the unused medical supplies.

16. The method of claim 13, further comprising customizing the pick list for each procedure based on preference data for that procedure stored in the at least one data storage device.

17. The method of claim 13, wherein the at least one location includes a first location where at least one of the predictable medical supplies is picked based on the pick list.

18. The method of claim 17, wherein the at least one location includes a second location where at least one of the unpredictable medical supplies is picked before the procedure.

19. The method of claim 18, wherein the at least one location includes a third location where at least one of the unpredictable medical supplies is picked during the procedure.

20. The method of claim 15, further comprising updating patient information based on at least one of the pick list and the unused medical supplies after the procedure is complete.
